(19) United States
(12) Patent Application Publication
(10) Pub. No.: US 2024/0000698 A1
(43) Pub. Date: Jan. 4, 2024

(54) COMPOSITION COMPRISING FERMENTED EXTRACT FROM PINE FOR PREVENTING SKIN WRINKLE, IMPROVING SKIN BARRIER FUNCTION, PREVENTING HAIR LOSS, PROMOTING HAIR GROWTH AND IMPROVING HAIR HEALTH AND METHOD FOR MANUFACTURING THE SAME

(71) Applicant: TaedamBio Co., Ltd., Gangneung-si (KR)

(72) Inventor: HEE SOOK KIM, Gangneung-si (KR)

(21) Appl. No.: 17/856,407

(22) Filed: Jul. 1, 2022

(30) Foreign Application Priority Data
Jun. 30, 2022 (KR) 10-2022-0080214

(51) Int. Cl. A61K 8/9767; A61K 8/9728; A61Q 19/08; A61Q 7/00

(52) U.S. Cl. CPC A61K 8/9767; A61K 8/9728; A61Q 19/08; A61Q 7/00

(57) ABSTRACT

Provided are a composition containing fermented pine extract that prevents skin wrinkles, improves skin barrier function, prevents hair loss, promotes hair growth and improves hair health, and a method for manufacturing the same. According to an embodiment of the present invention, a method for manufacturing a composition containing fermented pine extract that prevents skin wrinkle, improves skin barrier function, prevents hair loss, promotes hair growth and improves hair health is provided. The method includes the steps of extracting extract from a pine tree, producing pine extract by processing the extract, and producing fermented pine extract by fermenting the pine extract.

### CROSS REFERENCE TO RELATED APPLICATION

The present application claims priority to Korean Patent Application No. 10-2022-0080214, filed Jun. 30, 2022, the entire contents of which is incorporated herein for all purposes by this reference.

### BACKGROUND OF THE INVENTION

### Field of the Invention

The present invention relates to a composition containing fermented pine extract that prevents skin wrinkles, improves skin barrier function, prevents hair loss, promotes hair growth and improves hair health, and a method for manufacturing the same,

### Description of the Related Art

The moisture present in the stratum corneum of the epidermis makes the skin elastic and soft, and it is known that moisture content of 10% or more is essential to maintain the elasticity of the stratum corneum. Hydration of the skin is determined by a water loss equilibrium in the epidermis by the water transport due to osmotic pressure from the epidermis to the dermis and water evaporation. The level of moisture in the epidermis is highly dependent on keratinocyte intercellular lipids, corneodesmosom, and natural moisturizing factor (NMF). Since the dermis is mainly composed of collagen and elastin fibers to support the skin, when a problem occurs in the dermis, wrinkles are formed and skin elasticity is lost, leading to skin aging. In particular, collagen is known to play the most important role in skin regeneration, skin moisture content, wound healing, wrinkle treatment, and the like. Among them, type 1 collagen is known as the main component of connective tissue in the dermis. Hair loss is a phenomenon in which a lot of hair falls out abnormally, and the causes of hair loss include heredity, nutritional disorders, abnormal sebum secretion, hormones, stress, diseases, drugs, etc., and the hair loss is classified into non-scarring hair loss and scarring hair loss.

### SUMMARY OF THE INVENTION

Accordingly, the present invention provides is to provide a composition for cosmetic, food or pharmaceutical use that increases the expression of type 1 collagen by using a fermented extract derived from pine, which is a natural material, to prevent skin wrinkles, improve skin barrier function, and improves a main factor, which alleviates acid-base stimulation and facilitates nutrient supply, so that hair loss can be prevented, hair growth can be promoted and hair health can be improved.

According to one aspect of the present invention, a method for manufacturing a composition containing fermented pine extract that prevents skin wrinkle, improves skin barrier function, prevents hair loss, promotes hair growth and improves hair health is provided. The method includes the steps of extracting extract from a pine tree, producing pine extract by processing the extract, and producing fermented pine extract by fermenting the pine extract.

The step of extracting extract from a pine tree may include the steps of producing a pulverized product by drying and pulverizing the pine tree, and extracting the extract from the pulverized product by at least one of hot water extraction and solvent extraction.

In the step of producing a pulverized product, the pine tree may be cut to a set size, dried in a shade, and then pulverized.

The step of producing pine extract may include the step of collecting supernatant by reflux extraction of the extract and centrifugation after cooling the extract.

The step of producing pine extract may further include the steps of producing a concentrated solution by filtering the supernatant and then concentrating the supernatant under reduced pressure, and producing the pine extract by adding a solvent to the concentrated solution.

The step of producing fermented pine extract may include the steps of sterilizing the pine extract to remove toxicity, and fermenting the sterilized pine extract with yeast.

The yeast may include at least one of saccharomyces cerevisiea, tannase from aspergillus ficuum, lactobacillus buchneri, leuconostoc mesentorios, and bifidobacteria sp.

In the step of fermenting with yeast, the step of diluting the fermented pine extract to a set concentration, sterilizing the pine extract and fermenting with yeast may be repeatedly performed according to a set number of times.

The step of formulating the fermented pine extract for cosmetic use, or encapsulating for food or pharmaceutical use may be further included.

According to the present invention, there may be provided a composition for cosmetic, food or pharmaceutical use containing fermented pine extract, which is effective for preventing skin wrinkles, improving skin barrier function, preventing hair loss, promoting hair growth and improving hair health.

### DETAILED DESCRIPTION OF THE INVENTION

Hereinafter, a composition containing fermented pine extract that can prevent skin wrinkles, improve skin barrier function, prevent hair loss, promote hair growth and improve hair health, and a method for manufacturing the same according to the present invention will be described in detail. First, a composition according to an embodiment of the present invention will be described.

The composition according to an embodiment of the present invention (hereinafter referred to as composition) may contain fermented pine extract. Here, a pine tree may include pinus thunbergii, and the pinus thunbergii is a species of the genus of pine tree having black stems and branches, and may be named as Gomsol, Sussol, or Wansol in Korean.

The pine extract according to an embodiment of the present invention (hereinafter referred to as pine extract) may include those obtained by extraction treatment of a pine tree, and all formulations that can be formed using the same.

In addition, the pine extract may be one extracted from at least any one of bark, woody part, branch, fruit and leaf of a pine tree.

The pine extract may contain proanthocyanidins, or may contain any one of alpha-pinene, beta-pinene, beta-thujene, and myrcene. Here, alpha-pinene and myrcene may have an antioxidant effect, and alpha-pinene, beta-pinene, beta-thujene, and myrcene may have an immunity enhancing effect.

The pine extract may be in a state in which toxicity is removed from the extracted ingredients, and thus the stability of a composition may be secured.

The fermented pine extract according to an embodiment of the present invention (hereinafter referred to as fermented pine extract) may be prepared by fermenting the above-described pine extract. More specifically, the fermented pine extract may be prepared by fermenting the pine extract with yeast.

By including the fermented pine extract, the composition may have a better pharmacological effect compared to the composition containing unfermented pine extract. For example, the composition may contain fermented pine extract as an active ingredient to have physiological activity and biosorption capacity.

By including the fermented pine extract, the composition can suppress skin oxidation and skin inflammation. In addition, the composition contains the fermented pine extract to produce various effects such as supplying moisture to the stratum corneum of the skin epidermis, preventing skin wrinkles, improving the skin barrier function, preventing hair loss and promoting hair growth, and improving hair health.

When the composition is applied to the epidermis of the skin, such as an affected area or scalp, effects such as skin moisturizing, immunity enhancement, antioxidant, anti-aging, anti-cancer, photo-aging, anti-inflammatory, antibacterial, and pruritus relief may occur.

The composition may include the fermented pine extract in an amount of 0.001 to 30% by weight, preferably 0.1 to 20% by weight, based on the total weight of the composition.

The pine extract may exhibit excellent water retention ability, and accordingly, the composition containing the pine extract may be used for skin moisturizing.

The composition can exhibit the above-described effects by being used not only for skin care or hair care, but also for foods such as health functional foods and pharmaceuticals. When the composition is provided for hair care, it may be provided in the form of any one of a hair nourishing lotion, a scalp treatment, a hair tonic, a hair conditioner, a hair lotion, a hair shampoo, a hair conditioner, a hair treatment, and a hair cream.

Hereinafter, a method for manufacturing a composition according to an embodiment of the present invention will be described, but descriptions of contents overlapping with those described for the above-described pine extract, fermented pine extract, and composition will be omitted.

According to this embodiment, there is provided a method for manufacturing a composition including the steps of extracting extract from a pine tree, producing pine extract by processing the extract, and producing fermented pine extract by fermenting the pine extract.

According to this embodiment, there may be provided a composition for cosmetic, food or pharmaceutical use that contains fermented pine extract and prevents skin wrinkles, improves skin barrier function, prevents hair loss, promotes hair growth and improves hair health.

Hereinafter, each step of the method for manufacturing the composition according to the present embodiment will be described.

The step of extracting the extract from a pine tree may be performed by at least one of a hot water extraction method, a solvent extraction method, a cold extraction method, an ultrasonic extraction method, a filtration method, and a reflux extraction method. The type of the extraction solvent used to extract the pine extract may be water, a low alcohol, a polyhydric alcohol, a hydrocarbon-based solvent, or a mixture thereof.

The step of extracting the extract from a pine tree may include the steps of producing a pulverized product by drying and pulverizing the pine tree, and extracting the extract from the pulverized product by at least one of the hot water extraction and the solvent extraction.

In the step of producing a pulverized product, the pine tree may be cut to a set size and then pulverized after drying in a shade.

The step of producing the pine extract may include reflux extraction of the extract and centrifugation after cooling the extract to collect supernatant.

The suspended solid particles can be filtered and removed from the extract of the hot water extraction or the cold extraction. For example, the particles can be filtered using nylon and the like or filtered using a freeze filtration method, and the like, and then used as it is, or used by being dried with freeze drying, hot air drying, spray drying, and the like.

The step of producing the pine extract may further include the steps of producing a concentrated solution by filtering the supernatant and then concentrating the supernatant under reduced pressure, and producing the pine extract by adding a solvent to the concentrate solution. The step of producing the fermented pine extract may include the steps of sterilizing the pine extract to remove toxicity, and fermenting the sterilized pine extract with yeast.

The yeast may include at least any one of saccharomyces cerevisiea, tannase from aspergillus ficuum, lactobacillus buchneri, leuconostoc mesentorios, and bifidobacteria sp.

In the step of fermenting with yeast, the step of diluting the fermented pine extract to a set concentration, sterilizing the pine extract and fermenting with yeast may be repeatedly performed according to a set number of times.

The step of formulating the fermented pine extract for cosmetic use, or encapsulating for food or pharmaceutical use may be further included.

Accordingly, the composition may be prepared in a formulation selected from the group consisting of a solution, an external ointment, a cream, a foam, a nourishing lotion, a soft lotion, a pack, a soft water, an emulsion, a makeup base, an essence, a mist, a gel, a pack, a shampoo, a conditioner, a soap, a liquid detergent, a bath preparation, a sunscreen cream, a sun oil, a suspension, an emulsion, a paste, a gel, a lotion, a powder, a soap, a cleansing with surfactant, an oil, a powder foundation, an emulsion foundation, a wax foundation, a patch and a spray.

The composition may be encapsulated with one or more polymers selected from the group consisting of chitooligosaccharide, gelatin, pectin, polyethylene glycol, methyl cellulose, sodium carboxymethyl cellulose, hydroxymethylpropyl cellulose and ethyl cellulose.

Hereinafter, an example of manufacturing pine extract and fermented pine extract according to this embodiment, and test data related to the fermented pine extract prepared accordingly will be described.

<Method for Producing Pine Extract>

A pine tree (barks, xylem, leaves) was cut into pieces of 1 to 3 cm or less, respectively, and dried in the shade, and pulverized, and hot water extract and 70% EtOH extract were used in the preparation. For the hot water extract, 1.5 L of distilled water was added to 150 g of each part of the pine tree for 4 hours at 90° C., and for the 70% EtOH extract, 1.5 L of 70% EtOH was added to 150 g of each part of the pine tree for 4 hours at 60° C. The extract for each part of the pine tree was extracted under reflux twice for 4 hours, cooled, and then the solids were centrifuged at 4,000 rpm for 20 minutes using a centrifugal separator. The centrifuged supernatant was filtered twice for each part using a filter paper (0.45 μm filter paper, Advanted, Japan), and the filtrate was concentrated under reduced pressure in the condition of 40 to 60° C. and 90 rpm using a rotary vacuum evaporator. A pine extract was prepared using at least one solvent selected from purified water, ethanol, propylene glycol, and butylene glycol so that the concentrated solution under the reduced pressure and freeze-dried product were contained in % by weight. The solvent may be preferably purified water.

<Method for Producing Fermented Pine Extract>

The pine extract obtained by the above method was sterilized at 121° C. for 20 minutes. A mycelium disk was made with at least one mycelium selected from the group consisting of Saccharomyces cerevisiae, tannase from aspergillus ficuum, lactobacillus buchneri, leuconostoc mesentorios, and bifidobacteria sp. in the sterilized pine powder medium using a cork borer with a diameter of 8 mm. Then, 5 to 6 of these disks were inoculated into a 250 mL Erlenmeyer flask containing 100 mL of MCM medium, and incubated for 5 days in a Shaking Incubator (SI-4000R, JEIO TECH, Daejeon, Korea) at the conditions of a temperature of 25 to 30° C., a stirring speed of 120 rpm, and a aeration volume of 1.5 vm. After fermentation under optimal conditions for each fermentation strain, it was diluted with purified water 10 times the weight of the sample, and the same process was repeated 3 times to obtain the fermented pine extract.

<Cytotoxicity Test of Fermented Pine Extract>

This cytotoxicity test was performed to evaluate the cytotoxicity of the fermented pine extract obtained by the above method using CCD-986SK (Fibroblast) cells (Testing institution: Korea Testing and Research Institute, Test No.: TNK-2022-000240). Here, the cell line, CCD-986SK (source: ATCC CRL-1947) was used after being cultured in a humidified incubator at 37±1° C. in the presence of 5% CO₂ in DMEM medium supplemented with 10% FBS and 1% antibiotics (penicillin/streptomycin). The fermented pine extract was diluted to concentrations of 1, 5, 10, 20, and 30% in serum-free medium and used in the experiment. Here, as the serum-free medium, DMEM medium supplemented with 1% antibiotics (penicillin/streptomycin) was used. The cells were dispensed in a 96-well plate at 1×104 (100 μL/well) and cultured at 37° C., 5% CO₂ incubator for 24 hours. The test substance was replaced with a serum-free medium diluted by concentration and incubated for 24 hours at 37° C., 5% CO₂ incubator. After washing twice with PBS, 200 μL/well of MTT solution (5 mg/mL, serum-free medium) was dispensed and incubated for 2 hours at 37° C., 5% CO₂ incubator. After incubation, the medium was removed, 200 μL/well of DMSO solution was added, and after extraction for 20 minutes on a plate shaker, absorbance was measured using ELISA at a wavelength of 570 nm. When the cell viability was reduced to less than 70% of the sample-free group, it was determined that there was potential cytotoxicity, and the cell viability was calculated according to the following formula.

$$cell . viability ( \% ) = \frac{O . D . of . group . with . sample . at 570 nm}{O . D . of . group . without . sample . at 570 nm} \times 100 \qquad [ Formula 1 ]$$

Reviewing the results of the above test, as a result of treatment with 1%, 5%, 10%, 20% and 30% of the fermented pine extract, the cell viabilities of 102.71±2.49%, 76.48±1.69%, 68.86±1.76%, 65.55±3.13% and 11.18±0.68% were observed at each concentration, respectively. In conclusion, more than 70% cell viability was observed in the fermented pine extract at concentrations of 5% or less. If the cell viability was reduced to less than 70% of the cell viability of the sample-free group, it was determined to be potentially cytotoxic. Therefore, it was determined that the fermented pine extract had no cytotoxicity in the fermented pine extract at concentrations of 5% or less.

<Test of Application of Fermented Pine Extract on Human Body>

This test was conducted to evaluate the safety of the test product by skin patch of the fermented pine extract obtained by the above method through a human application test (Testing institution: Korea Testing & Research Institute, Test No.: TEK-2022-000139). The test was conducted with 31 adult males and females aged 20 to 59 years who did not have acute or chronic physical diseases including skin diseases as test subjects. In the condition in which 20 to 24° C. temperature and the humidity of 40 to 60% R.H. were maintained, the measurement site, which was back of the test subject, was washed with purified water, dried naturally for 5 minutes. Then, the IQ chamber in which 20 μL of the test product was dripped was fixed to the measurement site and a closed-patch test was conducted for 24 hours. Then, the IQ chamber was removed and the photographs were taken of the test product application site 30 minutes after the patch removal and 24 hours, and the degree of skin reaction was evaluated by a researcher. The visual evaluation of the test site was conducted according to the determination criteria of the International Contact Dermatitis Research Group (ICDRG). After the visual evaluation, the average skin reactivity for each test site was calculated using the following formula, and based on the calculated skin reactivity, the degree of skin irritation was determined according to the table below.

$$skin . reactivity = \frac{Σ ( Score \times No . of . Responders )}{4 ( Max . score ) \times No . of . evaulation \times N ( TotalSubjects )} \times 5 \qquad [ Formula 2 ]$$

TABLE 1

| Skin irritation index | Criteria |
|---|---|
| 0.00 to 0.75 | Non-irritation |
| 0.76 to 1.50 | Light-irritation |
| 1.51 to 2.50 | Mild-irritation |
| 2.51 to 4.00 | Medium-irritation |
| 4.01 to 5.00 | Strong-irritation |

Reviewing the results of the above test, a total of 31 subjects was tested, with an average age±standard deviation of 45.8±6.5 years old. By age group, there were 5 people in their 30s, 14 people in their 40s, and 12 people in their 50s. By skin type, 8 people were dry type, 2 people were oily type, 5 people were neutral type, and 16 people were combination type. As a result of the determination, the skin reactivity was 0.00, which was determined to be in the non-irritation category. During the use of the test product, no skin-related adverse events were reported and no adverse events were reported. In conclusion, fermented pine extract was determined as a product in the non-irritation category.

<DPPH Free Radical Scavenging Activity Measurement Test of Fermented Pine Extract>

The free radical scavenging activity measurement test was conducted using 2,2-diphenyl-1-picrylhydrazyl (DPPH) to evaluate the free radical scavenging activity contained in active oxygen, which is one of the antioxidant effects on the fermented pine tree extract obtained by the above method (testing institution: Korea Testing and Research Institute, test number: TNK-2022-000242). The fermented pine extract was prepared at 5, 10, 20, and 30% concentrations using ethanol, and as a positive control material, L-Ascorbic acid was weighed, and was prepared at concentrations of 0.0005, 0.001, 0.0015, and 0.002% using distilled water. Ethanol was used to prepare DPPH at concentrations of 0.2 mM. 100 μL of the preparations for each concentration were dispensed into a 96-well plate, 0.2 mM DPPH and 100 μL of Ethanol were added, and reacted at room temperature for 30 minutes in a light-shielding state, and then absorbance was measured at 517 nm wavelength. The DPPH free radical scavenging activity (%) was calculated using the following formula. Each experimental result was measured three times to calculate the mean and standard deviation, and the confidence interval was set at 95%.

$$DPPH . free . radical . scavenger . activity ( \% ) = ( 1 - \frac{A}{B} ) \times 100 \qquad [ Formula 3 ]$$

$$( A : Absorbance of test substance / B : absorbance of non - test substance )$$

Reviewing the results of the above test, the measured DPPH free radical scavenging activities were 73.52%, 91.86%, 93.06% and 93.69% for the test substances, the fermented pine extract at concentrations of 5%. 10%, 20% and 30%, respectively. The free radical scavenging activities were 17.86%, 37.20%, 57.25% and 77.39% for the positive control substance at concentrations of 0.0005%, 0.001%, 0.0015% and 0.002%, respectively, and as a result of calculating the IC₅₀ (concentration representing 50% activity), it was calculated at concentrations of 0.0013%. In conclusion, from the above results, in the DPPH free radical scavenging activity measurement test, it was determined that the fermented pine extract, a test substance, exhibited the DPPH free radical scavenging activity at concentrations of 5%, 10%, % and 30%.

What is claimed is:

1. A method for manufacturing a composition containing fermented pine extract that prevents skin wrinkle, improves skin barrier function, prevents hair loss, promotes hair growth and improves hair health, the method comprising the steps of:
extracting extract from a pine tree;
producing pine extract by processing the extract; and
producing fermented pine extract by fermenting the pine extract.

2. The method according to claim 1, wherein the step of extracting extract from a pine tree comprising the steps of:
producing a pulverized product by drying and pulverizing the pine tree; and
extracting the extract from the pulverized product by at least one of hot water extraction and solvent extraction.

3. The method according to claim 2, wherein in the step of producing a pulverized product, the pine tree is cut to a set size, dried in a shade, and then pulverized.

4. The method according to claim 1, wherein the step of producing pine extract comprises the step of collecting supernatant by reflux extraction of the extract and centrifugation after cooling the extract.

5. The method according to claim 4, wherein the step of producing pine extract further comprises the steps of:
producing a concentrated solution by filtering the supernatant and then concentrating the supernatant under reduced pressure; and
producing the pine extract by adding a solvent to the concentrated solution.

6. The method according to claim 1, wherein the step of producing fermented pine extract comprises the steps of:
sterilizing the pine extract to remove toxicity; and
fermenting the sterilized pine extract with yeast.

7. The method according to claim 6, wherein the yeast comprises at least one of saccharomyces cerevisiea, tannase from aspergillus ficuum, lactobacillus buchneri, leuconostoc mesentorios, and bifidobacteria sp.

8. The method according to claim 6, wherein in the step of fermenting with yeast, the step of diluting the fermented pine extract to a set concentration, sterilizing the pine extract and fermenting with yeast is repeatedly performed according to a set number of times.

9. The method according to claim 1, further comprising the step of formulating the fermented pine extract for cosmetic use, or encapsulating for food or pharmaceutical use.
